(19) United States
(12) Patent Application Publication
(10) Pub. No.: US 2013/0089856 A1
(43) Pub. Date: Apr. 11, 2013

(54) METHOD FOR IN VITRO DETECTION OF ATAXIA TELANGIECTASIA HEALTHY CARRIERS AND RELATED KIT

(71) Applicants: Silvia Soddu, Roma (IT); Enrico Cundari, Roma (IT); Luciana Chessa, Roma (IT); Andrea Prodosmo, Roma (IT)

(72) Inventors: Silvia Soddu, Roma (IT); Enrico Cundari, Roma (IT); Luciana Chessa, Roma (IT); Andrea Prodosmo, Roma (IT)

(21) Appl. No.: 13/522,642

(22) PCT Filed: Jan. 20, 2011
(86) PCT No.: PCT/IT2011/000018

§ 371 Date: Dec. 14, 2012

(30) Foreign Application Priority Data
Jan. 20, 2010 (IT) RM2010A000015

(51) Int. Cl. G01N 33/68

(52) U.S. Cl. CPC G01N 33/6896
USPC 435/6.1; 435/7.1

(57) ABSTRACT

The present invention concerns a method for in vitro detection of ataxia telangiectasia healthy carriers by determination of cell percentage wherein p53 is delocalized from centrosome during the mitosis.

The present invention concerns a method for in vitro detection of ataxia telangiectasia healthy carriers and related kit. Particularly, the present invention concerns a method for in vitro detection of ataxia telangiectasia healthy carriers by determination of percentage of cells wherein p53 is delocalized from centrosome during mitosis.

Telangiectasia Ataxia (AT) is an autosomal recessive inherited multisystemic disease resulting from omozigosys mutation of ATM gene (Ataxia Telangiectasia Mutated) and characterized by progressive neuronal degeneration mainly occurring through cerebellar ataxia, oculocutaneous telangiectasia, radiosensitivity and immunodeficiency resulting in predisposition to development of relapsing infections and tumours (Boder, 1985). Generally the death occurs during II/III life decades and it is due, by 80-90% of the cases, to neurological degeneration and by 10-15% of the cases to malignant lymphomas. Effective therapies for resolution treatment of the disease are not available. Symptomatic therapies comprise physiotherapy, language therapy and treatment of the infections and pulmonary complications. In tumour case, the use of x-ray and chemotherapy must be used carefully, because of the patient organism susceptibility to radiations.

At cellular level high sensibility to ionizing radiations and radiomimetic compounds occurs as a result of inability to activate checkpoints of cell cycle, chromosome instability and higher sensibility to apoptosis (Taylor et al., 1975). At gene level, AT is caused by ATM gene mutation and/or inactivation. Said gene, occurring on 11q22-23 chromosome, maps near 150 kb, comprises 66 hexons and it is among the largest up to now known genes. Actually more than 600 different mutations have been detected which, as to 80% of the patients, result in the expression of truncated ATM protein.

The disease occurs among all the populations. The incidence in USA people has been estimated as 1/40.000 alive born people (Swift et al., 1986). The frequency of healthy carriers is estimated as 0, 5-2% in whole population. The Registro Italiano per I'Atassia Telangiectasia (RIAT) extrapolated a theoretical frequency of the disease of 1/7090 conceptions and a frequency of healthy carriers from 1.69 to 3.43% in Italian population (Chessa et al., 1994).

The diagnosis is problematic; it is carried out about during third/fourth age year based on clinical evaluation and results of same laboratory tests (blood levels of alphaphetusprotein and immunoglobulins, increased and reduced, respectively, for the majority, approximately 95%, of AT affected subjects, karyotype analysis for the possible presence of characteristic translocations and analysis for susceptibility to ionizing radiations, increased for AT affected subjects and, partially, for healthy carriers). Diagnostic confirmation is obtained by means of molecular analysis (semiquantitative analysis of ATM protein levels by Western blot and ATM gene sequencing).

If on one hand the clinical observations and described laboratory analyses in combination allow the diagnosis of disease and detection, for families of affected subjects, of the healthy carriers of ATM mutations, to be carried out, the available tests, indeed, do not allow, a screening of the whole population. In fact, all the currently used techniques for the diagnosis or are not specific or need specialized laboratories, very long time to be carried out and elevated costs. Due to these factors an appropriated genetic advice is possible only for families wherein an AT affected subject is already present.

Currently used techniques for AT diagnosis need, for most cases, at least 6 weeks for culture stabilization of a lymphoblastoid cell line for each patient on which during next 2-4 weeks subsequent analyses will be carried out, that is: a) semiquantitative analysis of ATM protein levels by Western blot; b) analysis for susceptibility to ionizing radiations; and/or c) sequencing of ATM gene. In addition to require very long times and very high cost, these analyses suffer from further limitations.

The semiquantitative analysis of ATM protein levels suffer from two significant drawbacks. The first, technical, results from the fact that ATM protein is poorly expressed within cells of peripheral blood and thus the test must be carried out on a stabilized cell line of under test subject. This fact in addition of being time consuming, requires the availability of know-how specific analysis laboratory for stabilization and maintenance of cell cultures. The greatest difficulty results from the fact that in AT subjects ATM protein is absent in approximately 80% of patients, present at residual amounts (from 2 to 30%) in 18% of patients, and present at near normal amounts in approximately 2-3% of affected subjects. Accordingly not all the healthy carriers will have 50% of ATM protein; some will have levels near 100%, thus the semiquantitative determination of ATM protein levels results in a not reliable test as a routine diagnosis, particularly for healthy carriers.

Analysis for susceptibility to ionizing radiations requires, as above said test, a culture stabilized lymphoblastoid cell line for each patient or fibroblasts from subcutaneous biopsy and immortalized by SV40 infection or telomerase transduction. Said test, being based on hypersensitivity to ionizing radiations occurring in AT patients and, in lesser extent, also in heterozygotes, is not highly specific and is informative when associated to clinical data. It is therefore useful, in addition to diagnostic confirmation for AT patients, to study obliged carriers (parents of AT patients) and their relatives, but the identification accuracy of healthy carriers does not exceed 85% making this approach not usable for screening of a whole population.

Sequence analysis of ATM gene is the most reliable test, both for homozygosis and heterozygosis diagnosis. However it is very expensive (approximately 3000 Euros for each test) and requires the availability of a laboratory workers with a molecular biology and gene sequencing specific know-how. Therefore said test is highly specific, but high cost and embodiment difficulty thereof limit a large scale use.

In addition to limitations relating to genetic advice, the unavailability of a test for detection of ATM mutation healthy carriers is considered a focal point of biomedical research since such subjects seem to have an increased susceptibility to ionizing radiations and higher predisposition to develop cardiovascular pathologies and tumours. The healthy carriers do not display apparent phenotypic anomalies and therefore are not recognizable in whole population. Unfortunately, this detection failure results in remarkable limits like the inability to detect a pair at risk of generating AT affected subjects; the inability to verify, on statistically significant scale, the risk of healthy carriers, more sensitive to ionizing radiations than normal subjects, to develop some types of tumours (as for example breast and gastric carcinoma) and display higher side-effects as a result of diagnostic or therapeutic treatments requiring the use of ionizing radiations or radio-mimetic substances; the inability, when above said studies result in greater sensibility indications, to carry out screening on the whole population.

In particular, Swift showed a remarkable increase of breast tumours in feminine sex relatives of AT patients (Swift et al., 1987), while the data collected in Italy allowed to point out a remarkable increase of relative risk for gastric tumour in AT families compared to normal population (Chessa and Fiorilli, 1993). Studies carried out on subjects from breast tumour affected families showed that ATM germinal mutations confer to carriers a 2.37-2.7% relative risk to develop this cancer (Renwick et al, 2006; Paglia et al., 2009). Moreover, it has been recently showed that the defect of ATM protein or proteins regulated by the same contributes to unsuccessful repair of DNA double ruptures (Doksani et al., 2009). In addition to these observations there is also the fact the healthy carriers of AT mutations are more susceptible to ionizing radiations and consequently the related risk to develop tumours could be increased by diagnostic preventive procedures (for example mammography).

In the light of above, it is therefore apparent the need to provide a new method for detection of ataxia telangiectasia healthy carriers that overcomes the disadvantages of known art methods being particularly sensitive, specific, fast and economic.

Centrosomes are cytoplasmic organelles playing a key role both as to cytoskeleton organization in interphase cells and bipolar formation of mitotic fuse one during cell division. Recently a new function of centrosomes for integration of cellular response to genotoxic damage and proliferation stop signals has been detected. This new function is named “centrosome checkpoint” and various proteins responsible of more classic mechanisms (i.e. better studied) of response to DNA damage are involved, like Chk2, ATM and ATR kinases or oncosuppressor p53. During studies carried out by the inventors aimed to characterize the role of p53 in response to drugs suitable to inhibit the formation of mitotic fuse, it has been found that, during mitosis, p53 is localized within centrosome and phosphorylation of aminoacid residue serine 15 (p53Ser15) by ATM kinase is indispensable for such localization. After the localization within centrosome, the phosphorylation of p53Ser15 is eliminated (de-phosphorylation) and cellular mitosis can continue up to successive G1 phase and again resume the cellular cycle. In the presence of agents suitable to disaggregate microtubules and inhibit the formation of the mitotic fuse (for example, nocodazole and taxol), p53 is no longer localized within centrosomes and remains phosphorylated in Ser15. This does not prevent the cells to exit from mitosis, but during successive G1 phase, p53 is highly stabilized and induces the stop of the cellular cycle. These data suggest that the activation of p53 by ATM as a response to damages of mitotic fuse is regulated by localization of said p53 within centrosome (Ciciarello et al., 2001; Tritarelli et al., 2004).

Further studies allowed to demonstrate that the delocalization of p53 from the centrosome occurs also without treatment, within lymphoblastoid cells of AT affected patients.

Result similar to those observed for AT patients have been obtained using ATM pharmacological inhibitors, like caffeine. Moreover, the inventors have been able to correct the defect of p53 localization within centrosome within lymphoblastoids of AT patients by introducing again the native gene, confirming that ATM presence and corrected activity thereof are necessary in order p53 can be localized within centrosome (Oricchio et al., 2006).

The physio/pathological role carried out by ATM proteins and p53 within centrosome, as well as the molecular mechanisms regulating the same, are still largely unknown and require further analyses and studies as those carried out by the inventors.

In the course of these studies, the authors of the present invention surprisingly have observed that, during the mitosis of lymphocytes from AT healthy carriers, the percentage of cells with p53 centrosomal delocalization is from 45 to 60%. This behaviour is not expected on the base of what up to now known since all the lymphocytes display the same genetic defect in heterozygosis. Therefore, from a functional point of view, a similar behaviour is expected for all the lymphocytes, gradually from normal to some pathological extent, as for example it occurs both for other biological manifestations used as test for AT diagnosis (see above reported example with reference to ATM protein percentage) and in other genetic autosomal recessive diseases. For example, albinism healthy carriers do not have a cute with 50% not pigmented area (Griffiths et al., 2000), cystic fibrosis healthy carriers have all functionally normal cells (Iolascon et al., 2005), beta-thalassemia healthy carriers do not have 50% of red blood cells normal and remaining 50% thalassemic, but a generic reduction of erythrocyte volume and haemoglobin content as a whole. On the contrary, the fact that in AT healthy carriers now it has been detected that an half of lymphocytes has a normal behaviour and the other half according to an apparent pathological way is a completely unexpected behaviour, resulting, for the first time, in the possibility to detect the healthy carriers according to a sure, not invasive, economic and fast way.

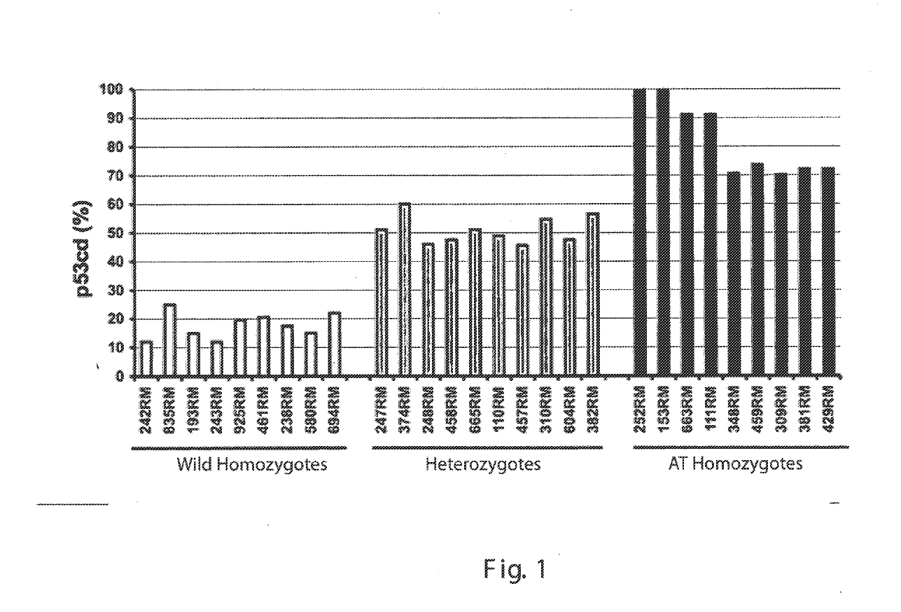

Particularly, the authors of the present invention have analyzed p53 mitotic localization in a significant number of human lymphoblastoid cell lines from AT patients, their parents and, when possible, healthy inter-familiar controls. As shown in FIG. 1, by measuring during the mitosis the percentage of cells with p53 centrosomal delocalization (thereafter named “p53cd”, that is p53 centrosomal delocalization), it has been observed p53cd in 70-100% of the cells from single lymphoblastoid populations derived from AT patients (black columns), this number drops to 10-25% for normal subjects (white columns), while it is between 45 and 60% of cells derived from parents of AT patients, that is obliged heterozygotes (grey columns). Therefore a remarkable discrimination margin for identification of heterozygotes exists. These results show, for the first time, that it is possible to distinguish, by p53cd determination, not only AT patients from normal subjects, but above all that heterozygotes from both homozygotes (wild type and AT) are distinguishable because display a consistently intermediate behaviour. In fact, FIG. 1 shows that within each genetic group the corresponding behaviour is highly reproducible. The percentage of p53cd has been calculated by verifying the overlapping of p53 and gamma-tubulin signals, gamma-tubulin being a centrosome specific marker, said signals obtained by recognition using specific antibodies and analysing, in at least 100 mitoses, the phase of the cellular cycle wherein p53 is localized within centrosomes. The failure of two signal overlapping is defined p53cd. Every single cell in mitosis possesses two centrosomes, but if one does not contain p53 it is enough in order said cell to be defined p53cd carrier. Therefore the determination is carried out considering cells and not centrosomes like single units. A percentage of 10% of p53cd means therefore that out 100 analyzed cells in mitotic phase, 10 have at least an aggregate (spot) of p53 that is not centrosomal co-localized while remaining 90 cells have p53 co-localized within both centrosomes. This behaviour is independent on the type of mutations (punctiform, deletion, insertions, reverse) that are found in cells from various AT healthy carriers, as reported in Table 1.

TABLE 1

| Obliged healthy | | |
|---|---|---|
| carrier | Mutation | p53cd (%) |
| 247RM | 1407 DEL 201/N | 51 |
| 374RM | 8283 DEL TC/N | 60 |
| 248RM | 1407 DEL 201/N | 46 |
| 458RM | 6572 INS 7/N | 47.5 |
| 665RM | 7408 T > G/N | 51 |
| 110RM | 755 DEL GT/N | 49 |
| 457RM | 8814 DEL GT/N | 45.5 |
| 310RM | IVS 37 + 2 T > C/N | 54.5 |
| 604RM | 8545 C > T/N | 47.5 |
| 382RM | 2250 G > A/N | 56.5 |
| 364RM | 7517 DEL 4/N | 50 |
| 365RM | 450 DEL 4/N | 56 |
| 536RM | 3993 INS 29 INTR 28/N | 52 |
| 559RM | 2718 DEL 4/N | 49 |
| 560RM | 2413 C > T/N | 51 |
| 773RM | 717 DEL CCTC/N | 52 |
| 906RM | 4396 C > T/N | 44 |
| K28RM | 5979 DEL 5/N | 52 |
| K33RM | 7517 DEL 4/N | 53 |
| K95RM | 2113 DEL T/N | 45 |

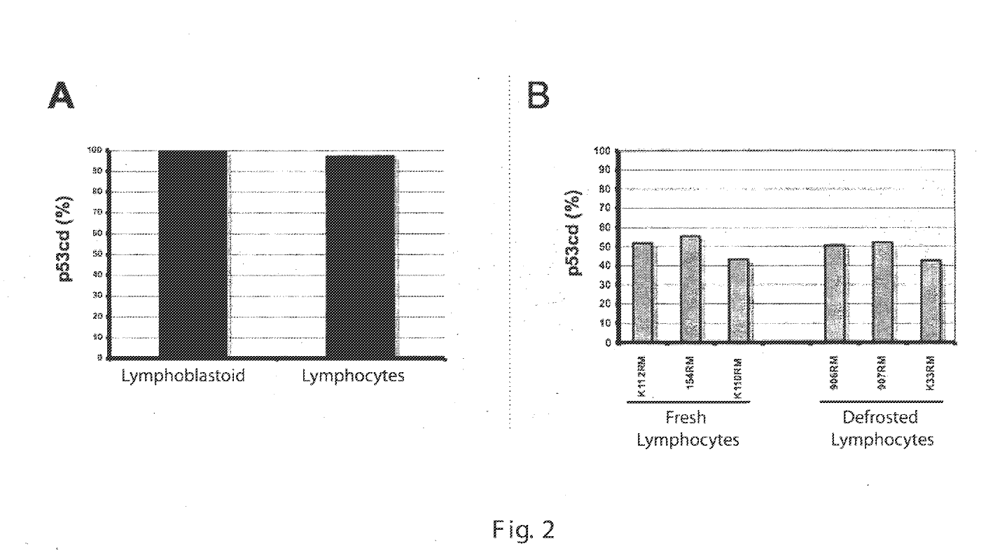

The observed behaviour occurs not only in lymphoblastoid cell lines, but it is perfectly reproducible in purified primary lymphocytes from peripheral blood and induced to culture proliferation (FIG. 2A). This result shows that observed phenotype is not from lymphocyte immortalization process and allows the p53cd determination directly in peripheral blood lymphocytes or any other tissue cell to be carried out, after only 2-3 days of proliferation culture stimulation, that is in a fast and economic way without the need of culture stabilization of lymphoblastoid cell lines. Moreover, we have not observed any difference when p53cd is measured in fresh or frozen peripheral blood lymphocytes (FIG. 2B). The percentage of p53cd has been measured in a lymphoblastoid cell line and peripheral blood lymphocytes drawn from the same patient after one year. The percentage remained unchanged indicating that nor effects resulting from lymphocyte culture immortalization nor phenotype substantial variations over the time occur. FIG. 2B shows p53cd measured percentage in fresh and liquid nitrogen frozen peripheral blood lymphocytes. The result show that the freezing process does not modify the p53cd percentage in thus analysed lymphocytes.

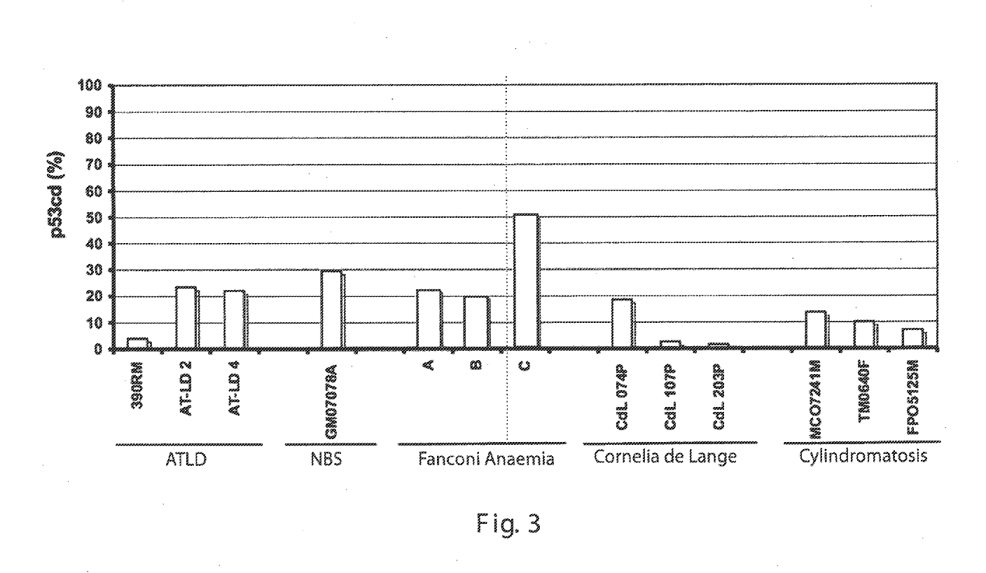

It has been, moreover, estimated the test specificity by analysis a set of lymphoblastoid cell lines from patients affected by pathologies relating to gene mutations of factors, as ATM proteins, related to DNA damage cell response. As shown in FIG. 3, no up to now analysed mutation causes p53cd at homozygosis level and, least of all, heterozygosis. Unique exception is represented by Fanconi anaemia wherein an AT heterozygosis similar picture occurs (approximately 50% of p53cd), but only in the event of FANC-C gene homozygosis. Being children affected by a severe form of congenital anaemia, the mix-up with AT healthy carriers is to be excluded. The percentage of p53cd has been measured on a set of lymphoblastoid cell lines from subjects affected by the following syndromes: AT-Like Disorder (ATLD), resulting from homozygosis mutation of MRE11A gene; Nijmegen Breakage Syndrome (NBS), also named AT V1 variant (AT-V1), resulting from homozygosis mutation of NBS1 gene; type A, type B and type C Fanconi anaemia, resulting from homozygosis mutation of FANC-A, FANC-B and FANC-C genes, respectively; Cornelia de Lange, resulting from homozygosis mutation of NIPBL, SMC1L1 or SMC3 genes; Cylindromatosis, resulting from mutation of CYLD gene.

It has been started the verification whether the test was suitable to confirming the data of increased risk for breast tumour. Aiming to said object, the test has been carried out, subject to authorization of Comitato Etico Istituto Tumori Regina Elena, on 80 subjects affected by breast carcinoma and 108 healthy subjects. 6 heterozygotes (7.5%) among patients and no heterozygotes among healthy individuals, suggesting that the test is suitable to confirm the significant increase of breast tumours originally observed by Swift among feminine sex consanguineous AT patients (Swift et al., 1987). ATM gene sequencing for test positive subjects confirmed the presence of mutations or F858L polymorphism associated with susceptibility to breast carcinoma (Izatt et al., 1999; Dörk et al., 2001; Rodriguez et al., 2002).

Moreover, in order to begin in understanding molecular mechanisms underlying the peculiar behaviour of AT heterozygote lymphocytes, i.e. the object of the present invention, we carried out a single cell cloning of lymphoblasts of cell line derived from 665RM obliged healthy carrier. Single sub-clones lost the distribution of p53cd for 50% and on the other hand shown p53cd typical for normal (10-15% of p53cd) or pathological (90-95% of p53cd) subjects, indicating that subject phenotype is not modified at each cell division, but more probably it is a constant characteristic of individual lymphocytes acquired during ontogenesis and lymphocyte development.

Based on above, the method according to the invention allows therefore the identification of AT healthy carriers by the determination of the loss of p53 (p53cd) mitotic localization within cells of a tissue biological sample like as for example peripheral blood lymphocytes. Currently, with the exception of whole ATM gene locus sequencing, which is an high sensitive and specific procedure but also very difficult and expensive, no method is available for this purpose. The method according to the invention on the contrary involves a quick analysis (72 hours for the purification and stimulation of lymphocytes, 1 working day for the staining and one/two hours for microscope readings); relative practice facility; cheapness (approximately 30 Euros for sample including worker cost); sensibility since heterozygotes display a quantitatively intermediate phenotype and, therefore, readily measurable, among normal and AT suffering subjects.

It is therefore a specific object of the present invention a method for in vitro detection of ataxia telangiectasia healthy carriers, said method comprising or consisting of the following steps:

a) determining the percentage of cells, in a tissue biological sample, preferably peripheral blood, wherein p53 is delocalized from centrosome for a statistically significant mitosis number for each sample; b) correlating said cell percentage value to a cell percentage range wherein p53 is delocalized from centrosome during the mitosis variable from 45 to 60% in ataxia telangiectasia healthy carriers. Since p53 can be localized or delocalized from centrosome, by calculating the localization percentage in a direct way p53 delocalization percentage is obtained. Therefore, a method for in vitro detection of ataxia telangiectasia healthy carriers by means of determination of p53 localization within centrosome is within the scope of the present invention. In this case the percentage of p53 localization will be from 75 to 90% for normal subjects, from 40 to 55% for healthy carriers, from 0 to 30% for AT affected subjects. The determination of cell percentage wherein p53 is delocalized from centrosome during the mitosis can be carried out by immunostaining technique. Particularly, the immunostaining can be carried out using anti-p53 antibodies, antibodies which bind a centrosomal protein and a DNA dye.

The centrosomal protein can be selected from the group consisting of gamma-tubulin, or other centrosomal protein as for example, LIS1; not characterized protein KIAA0841; GOGA3; not characterized protein C14orf94; HS90A; not characterized protein C14orf145; CE110; CEP41; CCR6; CP110; DYL1; DPOLN; TBA4A; GCP6; TBG1; GCP2; CE135; MYO1G; DCTN3; TBB5; 27 kDa centrosomal protein; GCP4; GCP3; 68 kDa centrosomal protein; 76 kDa centrosomal protein; 72 kDa centrosomal protein; 70 kDa centrosomal protein; CCD61; CLAP1; DC1L2; TBB4; 1433E; ALMS1; PRKAR2B; KAP3; LZTS1; NEDD1; AZI1; WDR8; PLK4; 170 kDa centrosomal protein; DC1L1; 78 kDa centrosomal protein; 164 kDa centrosomal protein; SCLT1; C5orf37 non characterized protein; CETN3; GCP5; WDR67; SASE; CCD52; KAP2; WD51A; PLK1; TBA1A; MDM1; 120 kDa centrosomal protein; AKNA; CDC2; CC123; 1433G; LRC45; CNTRB; 290 kDa centrosomal protein; ninein; PRKACA; DAB21P; SSNA1; K1731; PIBF1; 192 kDa centrosomal protein; 57 kDa centrosomal protein; 152 kDa centrosomal protein; WD51B; CSNK1D; PPP2R1A; PCM1; 63 kDa centrosomal protein; AKAP9; TBB2C; CCDC5; 97 kDa centrosomal protein; ODF2; CDK5RAP2; OFD1; NDE1; CKAP5; DCTN2; SDCCAG8; CEP250; CEP350; SFI1; DYHC1; EDC4; PCNT; not characterized putative protein ENSP00000371058; Irrcc1; DCTN1; NEK2; NME7; ACTR1A; CETN2; CROCC; MAPRE1; ANKRD26; UBA1; FAM29A; CENPJ; AKAP11; PARP-3; RAPGEF3; AURKA; PROCR; FSD1; CDH23; TTK; THG1; NUP85; CDC25B; LATS1; DCTN4; CCNB1; separin; KIF11; CAMK2A; CSNK1A1; JUB; MARK4; TXNDC9; CENPE; MADL1; TRIP11; BBS4; PML; KATNB1; SCYL1; cyclin A2; INCENP; PPP4C; subunits T-complex protein-1 epsilon sub-unit; CCDC5; CENPH; PPP4R1; PSKH1; PSY4; NPM1; NDEL1; MAPRE2; BIRC5; SAC3D1; mH2A1; Aurora-C; BNC1; CDC20; YPEL2; SSSCA1; Aurora B; hIFT20; CETN1; TBD; SPAST; CRMP1; TACC1; KIF15; PSEN1; myomegalin; RANBP1; CHEK2; TACC2; SPATC1; KATNA1; TTC8; DAP-5; RN19A; YPEL1; TEKT3; SYUG; protein 4.1; CDC5L; BRCA1; GIMA5; HMMR; CDC14A; TUBE1; PTP4A1; PLK3; RBGPR; MARE1; ID1; GDIB; PPP1R2P2; CDC27; ECM29; DISC1; MK11; TACC3; UXT; RB; p53; TALAN; STAT3; EVI5; PSMD1; CCND1; DCIL2; FAK2; DCX; MOS; SKP2; KPCB; MBD3; BBS7; SOCS1; NINL; KSYK; P85B; UCHL2; CRYAB; BBC3; RUVB; HSP7C; KRP1; SIK; BBS1; CHK1; CEP55; GIT1; HD; HSP72; PSMD3; MAP1A; TCPA; LCK; KPCT; HAP1; KIF14; STX8; TNKS1; MPK9; PDED4D; XPO1; FYN; CSPP1; MD2L1; BL1S2; RPGR1; CDK2; APC; PSME1; PPR11; MCRS1; COMT; CTNB1; S100B; GSK3A; PTN13; MPK3; MYO5A; MP2K2; PARG; KIFC1; WASF1; caspase precursor 3; TPP2; CC14B; KCC2B; ALS2; ANC; MAP2; CTND1; CLIC; PP1A; P55G; MPK8; CCNE1; CSK22; CALM; LATS2; PP1G; ROCK1; PSME2; KPCE; YPEL3; MMP14 precursor; ORC2; NUF2; XRCC1; FR1OP; NDC80; NDRG1; RGS14; KC1E; PPAR; FZR; RS27A; PSMD2; MCPH1; HBEGF precursor; IPP2; RFIP4; MAP2K1; FTCD; PP1B; PK2L1; DYL2; GSK3B; RUVB2; DAPK3; RAN; MAP6; SLMAP; MDM2; IBP4; RANB9; MAP4; P85A; PIM1; RASF1; MAP; TSC1; RABP1; HUNK; KCC2G; DYN2; PARP1; PARP2; CSK21; GPSM2; TAU; DLRB2; QN1; DNLI3; HSP70; ENOG; NUMA1; YPEL5; CENPF; SEPT9; CCNA2; IMB1; TOPB1; PAXI; BBS5; ANX11; 6PGD; CTCF; YPEL4; MPK10; CCNE2; RHOB precursor; DC1L1; AKT1; VINC; HDAC; CD2L2; DLRB1; BARD1; PRKN2; LIMK1; RPGR; MPK1; OGG1; MAPK5; MKKS; CUL1; PDE7A; PSN2; RFIP3; FAK1; PRS8; P11388 TOP2A 2 isoform; PNPH; inversin; ANR32; GLYAT; MARCS; CTAG2; MPH1; FEZ1; CPSF5; MLF1; NECD; PCGF5; GA45A.

Examples of anti-p53 antibodies that can be used are the following ones: human anti p53 mouse (clone DO-7) IgG_{2b} monoclonal antibody Nr. code M 7001 Dako, Anti-p53 (DO-7) IgG_{2b} monoclonal antibody Nr. sc-47698 Santa Cruz Biotechnology, Anti-p53 (DO-7) FITC IgG_{2b} monoclonal antibody Nr. sc-47698 FITC Santa Cruz Biotechnology, Anti-p53 (DO-7) PE IgG_{2b} monoclonal antibody Nr. sc-47698 PE Santa Cruz Biotechnology, Anti-p53 (B-P3) IgG_{2b} monoclonal antibody Nr. sc-65334 Santa Cruz Biotechnology, Anti-p53 (C-19) IgG polyclonal antibody Nr. sc-1311 Santa Cruz Biotechnology, Anti-p53 ab1101 ABCAM monoclonal antibody.

Among antibodies which bind centrosomal protein the following ones can be listed: Anti-γ-Tubulin polyclonal antibody, fraction of IgG antiserum, product No. T3559 Aldrich Sigma, Anti-γ-Tubulin (AK-15) polyclonal antibody, fraction of antiserum IgG Product No. T3220 Aldrich Sigma, Anti-γ-Tubulin (DQ-19) polyclonal antibody, antiserum fraction IgG product No. T3195 Aldrich Sigma, Anti-γ Tubulin (DTU-64) monoclonal antibody, purified immunoglobulin product No. T3950 Aldrich Sigma, ascitic fluid Anti-γ-Tubulin (GTU-88) monoclonal antibody, product No. T6557 Aldrich Sigma, Anti-γ-Tubulin (GTU-88) monoclonal antibody purified immunoglobulin product No. T5326 Aldrich Sigma, Anti-γ-Tubulin (C-11) IgG₂ₐ monoclonal antibody Nr. sc-17787 Santa Cruz Biotechnology, Anti-γ-Tubulin (C-20) IgG polyclonal antibody Nr. sc-7396 Santa Cruz Biotechnology, anti-γ-Tubulin (D-10) IgG_{2b} monoclonal antibody Nr. sc-17788 Santa Cruz Biotechnology, Anti-γ-Tubulin (D-10) PE IgG_{2b} monoclonal antibody Nr. sc-17788 Santa Cruz Biotechnology, Anti-γ-Tubulin (D-10) PE Alexa Fluor 405 IgG_{2b} monoclonal antibody Nr. AF405 sc-17788 Santa Cruz Biotechnology, Anti-γ-Tubulin (D-10) Alexa Fluor 488 IgG_{2b} monoclonal antibody Nr. sc-17788 AF488 Santa Cruz Biotechnology, Anti-γ-Tubulin (D-10) Alexa Fluor 647G2b monoclonal antibody Nr. sc-17788 AF647 Santa Cruz Biotechnology, Anti-γ-Tubulin (H-183) IgG polyclonal antibody Nr. sc-10732 Santa Cruz Biotechnology, Anti-γ-Tubulin (TU-30) IgG_{2b} monoclonal antibody Nr. sc-51715 Santa Cruz Biotechnology.

Among DNA dyes Hoechst 33258 or related bis-benzo imidazoles, propidium iodide, etidiium bromide, SYBR Green, YO-PRO-1 iodide, TO-PRO-3 iodide, TOTO-3 iodide, EvaGreen™ fluorescent DNA staining, anti-histone antibodies or other chromatin associated proteins can be mentioned.

The cells useful for determinations are selected from the group consisting of lymphocytes, fibroblasts, amniotic cells, stem cells, umbilical cord cells.

Particularly, the method according to the invention can comprise or consist of the following steps:
a) isolation of cells contained within a tissue biological sample, preferably peripheral blood;
b) possible stimulation of cell proliferation;
c) cell fixing and immunostaining of p53 and a centrosomal protein using an anti-p53 antibody, an antibody binding a centrosomal protein and a DNA dye, as for example the above reported antibodies and dyes; and
d) quantification of cell percentage wherein p53 is delocalized from centrosome during a statistically significant mitosis number for each sample, said percentage being from 45 to 60% for ataxia telangiectasia healthy carriers. The stimulation of cellular proliferation (phase b) is carried out only for non proliferating cells without stimulation.

The use of a kit for in vitro detection of AT healthy carriers by determination of cell percentage, in a tissue biological sample, wherein p53 is delocalized from centrosome, during a statistically significant mitosis number for each sample is a further object of the invention, said kit comprising or consisting of:
a) an anti-p53 antibody, as for example, human anti p53 mouse (clone DO-7) IgG_{2b} monoclonal antibody Nr. code M 7001 Dako, Anti-p53 (DO-7) IgG_{2b} monoclonal antibody Nr. sc-47698 Santa Cruz Biotechnology, Anti-p53 (DO-7) FITC IgG_{2b} monoclonal antibody Nr. sc-47698 FITC Santa Cruz Biotechnology, Anti-p53 (DO-7) PE IgG_{2b} monoclonal antibody Nr. sc-47698 PE Santa Cruz Biotechnology, Anti-p53 (B-P3) IgG_{2b} monoclonal antibody Nr. sc-65334 Santa Cruz Biotechnology, Anti-p53 (C-19) IgG polyclonal antibody Nr. sc-1311 Santa Cruz Biotechnology, Anti-p53 ab1101 ABCAM monoclonal antibody;
b) an antibody binding a centrosomal protein, for example gamma-tubulin, as for example, Anti-γ-Tubulin polyclonal antibody, fraction of IgG antiserum, product No. T3559 Aldrich Sigma, Anti-γ-Tubulin (AK-15) polyclonal antibody, fraction of antiserum IgG Product No. T3220 Aldrich Sigma, Anti-γ-Tubulin (DQ-19) polyclonal antibody, antiserum fraction IgG product No. T3195 Aldrich Sigma, Anti-8 Tubulin (DTU-64) monoclonal antibody, purified immunoglobulin product No T3950 Aldrich Sigma, ascitic fluid Anti-γ-Tubulin (GTU-88) monoclonal antibody, product No. T6557 Aldrich Sigma, Anti-γ-Tubulin (GTU-88) monoclonal antibody purified immunoglobulin product No. T5326 Aldrich Sigma, Anti-γ-Tubulin (C-11) IgG₂ₐ monoclonal antibody Nr. sc-17787 Santa Cruz Biotechnology, Anti-γ-Tubulin (C-20) IgG polyclonal antibody Nr. sc-7396 Santa Cruz Biotechnology, anti-γ-Tubulin (D-10) IgG_{2b} monoclonal antibody Nr. sc-17788 Santa Cruz Biotechnology, Anti-γ-Tubulin (D-10) PE IgG_{2b} monoclonal antibody Nr. sc-17788 Santa Cruz Biotechnology, Anti-γ-Tubulin (D-10) PE Alexa Fluor 405 IgG_{2b} monoclonal antibody Nr. AF405 sc-17788 Santa Cruz Biotechnology, Anti-γ-Tubulin (D-10) Alexa Fluor 488 IgG_{2b} monoclonal antibody Nr. sc-17788 AF488 Santa Cruz Biotechnology, Anti-γ-Tubulin (D-10) Alexa Fluor 647G2b monoclonal antibody Nr. sc-17788 AF647 Santa Cruz Biotechnology, Anti-γ-Tubulin (H-183) IgG polyclonal antibody Nr. sc-10732 Santa Cruz Biotechnology, Anti-γ-Tubulin (TU-30) IgG_{2b} monoclonal antibody Nr. sc-51715 Santa Cruz Biotechnology;
c) a DNA dye as for example Hoechst 33258 or related bis-benzo imidazoles, propidium iodide, etidiium bromide, SYBR Green, YO-PRO-1 iodide, TO-PRO-3 iodide, TOTO-3 iodide, EvaGreen™ fluorescent DNA staining, anti-histone antibodies or other chromatin associated proteins; and, possibly,
d) a cell proliferation stimulating agent as for example phytohemagglutinin, interleukines, cytokines, fibroblast growth factors, epithelial cell growth factors, hemopoietic stem cell growth factors and not, insulin or insulin-like growth factors.

The present invention now will be described by an illustrative, but not limitative, way according to preferred embodiments thereof, with particular reference to enclosed drawings, wherein:

FIG. 1 shows centrosome delocalized p53 percentage in wild type homozygotes, heterozygotes, AT homozygotes;

FIG. 2 shows centrosome delocalized p53 percentage in lymphoblasts and lymphocytes (panel A) and in fresh or frozen lymphocytes (panel B);

FIG. 3 shows centrosome delocalized p53 percentage in a set of lymphoblastoid cell lines from patients affected by the following syndromes: AT-Like Disorder (ATLD); Nijmegen Breakage Syndrome (NBS); type A, type B and type C Fanconi anaemia, Cornelia de Lange; Cylindromatosis.

### EXAMPLE 1

### Study of p53 Mitotic Localization in Human Lymphoblastoid Cell Lines Obtained from AT Patients or Healthy Parents Thereof

Lymphoblastoid cells are from Ataxia Telangiectasia affected patients and the whole ATM gene (AT homozygotes) thereof has been sequenced, heterozygotes are from parents of naturally obliged patients while wild type homozygotes are from patients not affected by this syndrome.

All the lymphoblastoid cell lines have been obtained from corresponding lymphocytes by immortalization using EBV virus infection.

All the cells (lymphocytes and lymphoblastoids) have been cultured in RPMI 1640 medium supplemented with 10% bovine serum and 2 μm β-mercapto ethanol at 37° C. in 5% CO₂. Lymphoblastoid cell lines do not need growth stimulation and after 24 h incubation have been harvested and the below described experimental protocol has been applied.

Experimental Protocol

### a) Isolation and Stimulation of Peripheral Blood Lymphocytes

Centrifugation of 5 ml of peripheral blood on Ficoll gradient or equivalent (2000 rpm for 20 min)

Lymphocytes collection at Ficoll and plasma interface

Two washings of lymphocytes with PBS (1000 rpm for 10 min)

Stimulation of lymphocytes (5×10⁵/ml) in culture medium in the presence of phytohemagglutinin (PHA 5 mg/ml) or equivalent, for 60 h at 37° C.

### b) Cell Fixing (Stimulated Peripheral Blood Lymphocytes or Lymphoblastoid Cell Lines)

Suspended cells (8×10⁵ cell/slide) centrifuged at 1000 rpm for 7 min

One PBS washing (1000 rpm for 7 min)

PBS re-suspended cells plated in 35 mm Petri dishes on 100 μg/ml poly-lysine treated cover-slips (c-s) (1 ml cell suspension/c-s) and then centrifuged at 2000 rpm for a few second and then gradually down to 1000 rpm for 7 min.

Drain away supernatant

Fixing in 3.7% formaldehyde (PBS diluted) for 10 min at room temp.

Fast PBS washing

0.25% PBS-Triton X-100 washing for 5 min

Fast PBS washing

Fixing in neat MetOH for 10 min at −20° C.

Fast PBS washing

Store in PBS at +4° C.

### c) p53 and γ-Tubulin Immunostaininq

Two 0.05% PBS-Tween 20 fast washings

3% BSA pre-incubation or equivalent (dissolved in 0.1% PBS-Triton X-100) for 30 min (40 μl/slide)

Fast 0.05% PBS-Tween 20 washing

Incubation with anti-p53 antibody and Anti-γ-tubulin in 1% BSA for 2 h at 37° C. (in humid room)

Three 0.05% PBS-Tween 20 fast washings+three 0.05% PBS-Tween 20 washings for 5 min

3% BSA pre-incubation (dissolved in 0.1% PBS-Triton X-100) for 15 min (40 μl/slide)

Fast 0.05% PBS-Tween 20 washing

Secondary antibody incubation in 1% BSA for 30 min at 37° C. in the dark (in humid room)

Three 0.05% PBS-Tween 20 fast washings+three 0.05% PBS-Tween 20 washings for 5 min

Counter-staining with 0.1 μg/ml DAPI in PBS or equivalent. Incubate for 1 min in the dark

Two PBS fast washings'+three PBS washings for 5 min

Slides on cover-slips with 6 μl of Vectashield or equivalent.

### d) Microscope Readings for p53 and γ-Tubulin Relative Localization

Evaluation of p53 and γ-Tubulin co-localization percentage, that is presence or de-localization of p53 from centrosome, in at least 100 mitotic cells for each sample. An adequately skilled worker can read up to approximately 100 mitotic cells/hour for a fair preparation of lymphocytes.

### BIBLIOGRAPHY

1. Boder. Ataxia-telangiectasia: Genetics, Neuropathology and Immunology of a Degenerative Disease of Childhood. Gatti and Swift New York: Alan R. Liss (pub.) 1985
2. Dörk T, Bendix R, Bremer M, Rades D, Klöpper K, Nicke M, Skawran B, Hector A, Yamini P, Steinmann D, Weise S, Stuhrmann M, Karstens J H. “Spectrum of ATM Gene Mutations in a Hospital-based Series of Unselected Breast Cancer Patients” Cancer Res. 2001 61:7608-15.
3. Chessa L, Lisa A, Fiorani O, Zei G. Ataxia-telangiectasia in Italy: genetic analysis. Int J Radiat Biol. 66 Suppl: S31-33 1994
4. Chessa and Fiorilli. Ataxia-Telangiectasia. NATO ASI Series, vol. H77, Gatti and Painter 1993
5. Ciciarello M, Mangiacasale R, Casenghi M, D'Angelo M, Soddu S, Lavia P, and Cundari E. p53 Displacement from Centrosomes and p53-Mediated G1 Arrest Following Transient Inhibition of the Mitotic Spindle. J Biol Chem 276:19205-19213, 2001.
6. Doksani Y, Bermejo R, Fiorani S, Haber J E, Foiani M. Replicon dynamics, dormant origin firing, and terminal fork integrity after double-strand break formation. Cell. 137:247-258., 2009
7. Griffiths A. J. F., Miller J. H., Suzuki D. T., Lewontin R. C. Gelbart W. M. “An introduction to Genetic Analysis” 2000 W.H. Freeman and Company
8. Iolascon, Gasparini P., Cocozza. “GENETICA MEDICA lineamenti” 2005 IDELSON-GNOCCHI
9. Izatt L, Greenman J, Hodgson S, Ellis D, Watts S, Scott G, Jacobs C, Liebmann R, Zvelebil M J, Mathew C, Solomon E. “Identification of Germline Missense Mutations and Rare Allelic Variants in the ATM Gene in Early-Onset Breast Cancer”. Genes Chromosomes Cancer. 1999 26:286-94.
10. Oricchio E, Saladino C, Iacovelli S, Soddu S, Cundari E. ATM is Activated by Default in Mitosis, Localizes at Centrosomes and Monitors Mitotic Spindle Integrity. Cell Cycle 5(1): 88-92, 2006.
11. Paglia L L, Laugé A, Weber J, Champ J, Cavaciuti E, Russo A, Viovy J L, Stoppa-Lyonnet D. ATM germline mutations in women with familial breast cancer and a relative with haematological malignancy. Breast Cancer Res Treat. 2009
12. Renwick A, Thompson D, Seal S, Kelly P, Chagtai T, Ahmed M, North B, Jayatilake H, Barfoot R, Spanova K, McGuffog L, Evans D G, Eccles D; Breast Cancer Susceptibility Collaboration (UK), Easton D F, Stratton M R, Rahman N. ATM mutations that cause ataxia-telangiectasia are breast cancer susceptibility alleles. Nat. Genet. 38:873-875, 2006
13. Rodriguez C, Vallès H, Causse A, Johannsdottir V, Eliaou J F, Theillet C. “Involvement of ATM Missense Variants and Mutations in a Series of Unselected Breast Cancer Cases” Genes Chromosomes Cancer. 2002 33:141-9.
14. Swift M, Morrell D, Cromartie E, Chamberlin A R, Skolnick M H, Bishop D T. The incidence and gene frequency of ataxia-telangiectasia in the United States. Am J Hum Genet. 39:573-583, 1986
15. Swift M, Reitnauer P J, Morrell D, Chase C L. Breast and other cancers in families with ataxia-telangiectasia. N Engl J. Med. 316:1289-1294, 1987
16. Taylor A M, Harnden D G, Arlett C F, Harcourt S A, Lehmann A R, Stevens S, Bridges B A. Ataxia telangiectasia: a human mutation with abnormal radiation sensitivity. Nature 258:427-429, 1975
17. Tritarelli A, Oricchio E, Ciciarello M, Mangiacasale R, Palena A, Lavia P, Soddu S, Cundari E. p53 Localization at Centrosomes during Mitosis and Postmitotic Checkpoint are ATM-Dependent and Require Serine 15 Phosphorylation. Mol Biol Cell 15:3751-3757, 2004.

1. A method for in vitro detection of ataxia telangiectasia healthy carriers, said method comprising:
a) determining a percentage of cells, in a tissue biological sample, wherein p53 is delocalized from centrosome for a statistically significant mitosis number for each sample thus obtaining a cell percentage value;
b) correlating said cell percentage value to a cell percentage range wherein p53 is delocalized from centrosome during the mitosis variable from 45 to 60% in ataxia telangiectasia healthy carriers.

2. The method according to claim 1, wherein the determining is carried out by immunostaining.

3. The method according to claim 2, wherein the immunostaining is carried out using anti-p53 antibodies, antibodies which bind a centrosomal protein and a DNA dye.

4. The method according to claim 3, wherein the centrosomal protein is gamma-tubulin.

5. The method according to claim 1, wherein the tissue biological sample is peripheral blood.

6. The method according to claim 1, wherein the cells are selected from the group consisting of lymphocytes, fibroblasts, amniotic cells, stem cells, umbilical cord cells.

7. The method according to claim 1, comprising:
a) isolating cells contained within a tissue biological sample;
c) fixing the isolated cells and immunostaining p53 and a centrosomal protein using an anti-p53 antibody, an antibody binding a centrosomal protein and a DNA dye; and
d) quantifying a cell percentage wherein p53 is delocalized from centrosome during a statistically significant mitosis number for each sample, said percentage ranging from 45 to 60% for ataxia telangiectasia healthy carriers.

8. A kit for in vitro detection of AT healthy carriers by determination of cell percentage, in a tissue biological sample, wherein p53 is delocalized from centrosome, during a statistically significant mitosis number for each sample, said kit comprising:
a) an anti-p53 antibody;
b) an antibody binding a centrosomal protein;
c) a DNA dye; and, possibly,
d) a cell proliferation stimulating agent.

9. The method according to claim 7, further comprising stimulating cell proliferation of the isolated cells.
